(19) United States
(12) Patent Application Publication
(10) Pub. No.: US 2009/0247966 A1
(43) Pub. Date: Oct. 1, 2009

(54) METHODS AND PRODUCTS FOR APPLYING STRUCTURED COMPOSITIONS TO A SUBSTRATE

(76) Inventors: Euen T. Gunn, Trenton, NJ (US); Glenn A. Nystrand, Lebanon, NJ (US); Kurt Schilling, North Caldwell, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/057,635

(22) Filed: Mar. 28, 2008

(51) Int. Cl. A61M 35/00

(52) U.S. Cl. 604/289

(57) ABSTRACT

Provided are methods of, and personal care products for, applying a structured composition to a body with reduced wet slip. The methods and products apply the composition such that upon application a plurality of channels are disposed between the composition and the substrate.

### FIELD OF INVENTION

The present invention relates to methods of applying a structured composition to a substrate and products comprising a structured composition for application to a substrate.

### DESCRIPTION OF THE RELATED ART

In the fields of personal care, home care, and other consumer products it is known to provide a variety of types of compositions in a variety of types of packaging. The compositions may be of various forms, e.g., powders, pastes, liquids, gels, creams, ointments, sticks, serums, among other forms. The compositions may be single-phase systems, such as solutions or multiple phase systems such as emulsions. The product form and number of phases may be selected for various reasons, e.g., phase stability, product aesthetics, and the like. Similarly packaging can take various forms, e.g., pumps, sprays, contact sticks, or bottles adapted to extrude the composition therethrough. The composition once expressed from the container is applied to one of various a substrates, e.g., a hard surface such as the floor of a bathroom; a soft surface such as carpeting; a exterior body part such as the skin, hair, nails, or eyes; or an internal body surface such as the oral cavity or mucosa.

Applicants have recognized that it may be desirable to improve the ability of a composition, once expressed from its container, to resist slipping across the substrate. For example, Applicants have recognized that it is undesirable for a body wash composition, once expressed from its container, to slip across wet skin and onto the shower floor before the user has an opportunity to rub the body wash across the skin.

In light of the above, applicants have recognized the need to develop methods and product, e.g. personal care products, that have reduced tendency to slip across substrates, such as, but not limited to a moist body surface.

### SUMMARY OF THE INVENTION

The present invention meets the aforementioned need and overcomes the disadvantages of the prior art. In particular, applicants have discovered that a significant reduction in wet slip of structured compositions on a substrate may be achieved by applying the composition to the substrate such that a plurality of channels are disposed between the composition and the substrate.

According to one aspect, the present invention provides a method of applying a structured composition to a body, said method comprising contacting a substrate on a body surface with a structured composition such that a plurality of channels are disposed between the composition and the substrate.

According to another aspect, the present invention comprises a personal care product comprising a container suitable to be held in a user's hand, the container comprising an orifice, wherein the orifice defines a shape that includes a plurality of protrusions and a plurality of indentations and a composition having a Yield Stress from about 1 Pascal (Pa) to about 1500 Pa. within said container, wherein the composition, when extruded through the orifice is suitable to conform sufficiently to the shape of the orifice to form an extruded portion of structured composition, the extruded portion of structured composition thereby comprising a plurality of contact surfaces for contacting a body surface and a plurality of channels intermediate the contact surfaces.

### BRIEF DESCRIPTION OF THE DRAWINGS

Examples of embodiments of the present invention will now be described with reference to the drawings, in which:

FIG. 1 is a top view of a structured composition applied to a substrate in accord with one embodiment of the invention;

FIG. 2 is a top view of a structured composition applied to a substrate in accord with one embodiment of the invention;

FIG. 3 is a view from the substrate-contacting side of a structured composition to be applied to a substrate in accord with one embodiment of the invention;

FIG. 4 is a perspective view a personal care product in accordance with an embodiment of the present invention, wherein a user is urging a composition from a container onto the user's skin;

FIG. 5 is a top view of an orifice of the container of FIG. 4;

FIG. 6 is a top view of the orifice of FIGS. 4-5, showing additional features thereof;

FIG. 7 is a perspective view of the orifice of FIG. 4 and the composition being urged through the orifice, further revealing an ability of the composition to “remember” the shape of the orifice;

FIG. 8 is an alternative orifice consistent with embodiments of the inventions described herein; and

FIG. 9 is a top view of an orifice of the prior art.

### DESCRIPTION OF PREFERRED EMBODIMENTS

All percentages listed in this specification are percentages by weight, unless otherwise specifically mentioned.

As noted above, applicants have discovered unexpectedly that undesirable slip across a moist or wet substrate such as a body surface may be reduced by contacting the substrate with the structured composition such that a plurality of channels are defined by, and disposed between, the structured composition and said substrate.

The applicants have further discovered that the reduction of wet slip may be reduced by employing a container suitable to be held in the hand of a user, the container including an orifice that defines a shape that includes a plurality of protrusions and a plurality of indentations, such that when a structured composition is extruded through the orifice, the extruded composition comprises a plurality of contact surfaces for contacting a body surface and a plurality of channels intermediate the contact surfaces.

More specifically, applicants have tested the wet slip and the weight-normalized wet slip (WSₙ) associated with structured compositions applied in a variety of manners, and having a variety of shapes as applied to the skin, via the Wet Slip Test as defined and described below. Applicants have discovered unexpectedly that compositions applied to a substrate in a manner such that a plurality of channels are formed between, and defined by, at least one surface of the substrate and at least a portion of the composition, tend to exhibit significantly reduced wet slip and WSₙ as compared to comparable compositions applied so as to form only one or no channels between the substrate and the composition.

For example, as shown in Tables 2 and 3, applicants have measured the wet slip associated with structured compositions applied in accord with the present invention as compared to wet slip of the same structured compositions applied in various other comparable methods that form less than two channels when the composition is applied to the substrate. As illustrated, the methods of the present invention tend to result in a wet slip and a WSₙ that is significantly less than other methods. In certain embodiments, the present methods tend to have a WSₙ that is at least two times to as much as ten times or more less than comparable methods. In certain preferred embodiments, the present methods tend to result in compositions that exhibit a WSₙ of less than about 5 inches per gram, more preferably less than about 4 inches per gram or less, even more preferably, less than about 3 inches per gram.

Any suitable substrate may be used in accord with the present methods. Examples of suitable substrates include surfaces of the body, such as but not limited to, skin, mucosal tissue, hair, nails, and the like. Preferably, the substrate used in the present method is a “wet substrate.” As used herein the term “wet substrate” or “moist substrate” refers generally to substrate comprising water on a surface thereof. Preferably, the wet substrate comprises water on a surface to which a composition of the present invention is to be applied in accord with the present methods. Any suitable substrate having water thereon may be a wet substrate in accord with the present invention. In certain preferred embodiments, the wet substrate of the present invention comprises wet skin, wet mucosal tissue, a wet ocular surface or combinations thereof. In certain more preferred embodiments, the wet substrate comprises wet skin.

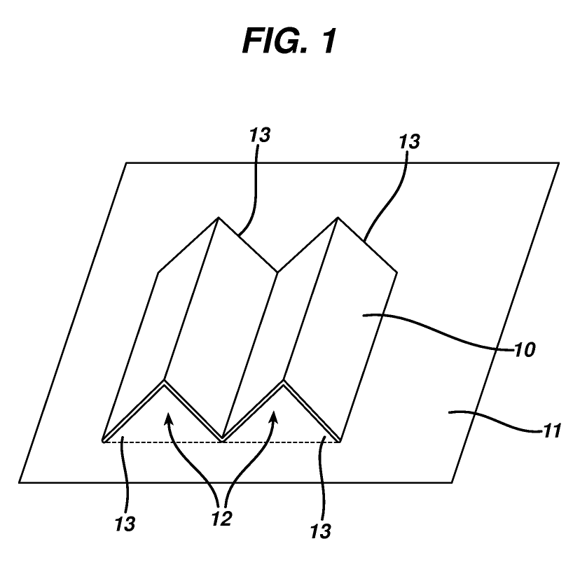

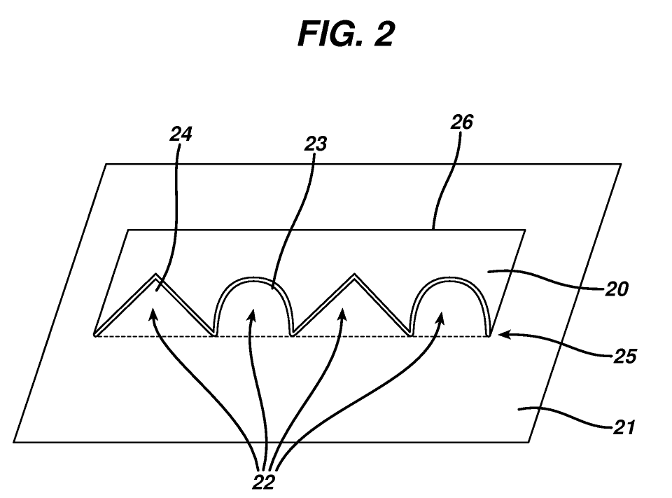

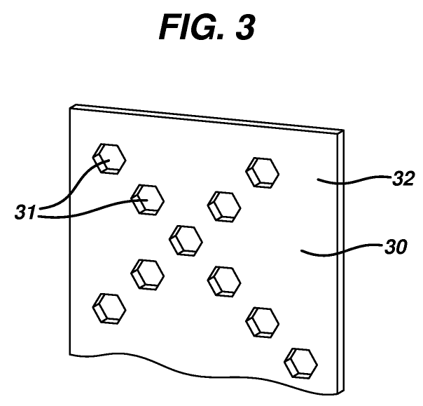

As used herein, the term “channel” refers to a space defined by and between the structured composition and a surface of the substrate comprising only air or gasses of the surrounding atmosphere in which the composition is applied to the substrate. A channel may be of any suitable shape and size and may be open to the surrounding atmosphere via one or more openings or may be closed to the surrounding atmosphere. The plurality of channels of the present methods may comprise any combination of two or more channels of the same or different size and/or shape. For example, FIG. 1 shows a structured composition 10 applied to a substrate 11 in accord with one embodiment of the present invention having a plurality of channels 12 of the same relative size and shape, including triangular openings to the atmosphere 13 at opposing ends of the applied composition and a triangular cross-section. FIG. 2 shows a view of an applied structured composition in accord with an embodiment of the present invention wherein a composition 20 and a substrate 21 define and comprise a plurality of channels 22, wherein some of the channels 23 have a relatively rounded cross section and alternating channels 24 have a triangular cross-section, and all of the channels are open at one end 25 and closed at the opposing end 26. FIG. 3 shows a structured composition 30 of one embodiment of the present invention having a variety of discreet shapes 31 imparted thereto such that upon application of the substrate-contacting side 32 of the composition to the substrate, the shapes 31 shall define closed channels between the composition 30 and substrate (not shown). In certain preferred embodiments, the plurality of channels comprise channels of substantially the same size and shape. As used herein, the term “substantially the same size and shape” means two or more channels wherein each of the various dimensions thereof (e.g. length, height, depth, cross-section diameter) as compared to the respective dimensions of the other channel(s) are the same or vary by less than 25%, preferably less than 10%, more preferably less than 5%. In certain preferred embodiments, the channels are open to the atmosphere. In certain preferred embodiments, the channels when applied have a height of from about 0.5 mm to about 5 mm, preferably from about 0.5 mm to about 3 mm, more preferably from about 0.75 mm to about 2 mm.

Any suitable number of two or more channels may be disposed between the composition and substrate in accord with the present methods. The number of protrusions and channels are not critical, but the inventors have found that at least two channels are required. In one embodiment, the boundary of the orifice 7 has from 2 to about 100 channels, more preferably from about 2 to about 20 channels, even more preferably from about 2 to about 10 and most preferably from about 3 to about 10 channels.

Any suitable methods for imparting a shape to the composition for forming the plurality of channels upon application to the substrate may be used in accord with the present methods. Examples of suitable methods of imparting a suitable shape to the composition include extruding the composition through an orifice, including through an orifice designed to impart grooves or shapes suitable for forming a plurality of channels as further described below. Other means for imparting a suitable shape to the composition include, for example, extruding the structured composition through a plurality of orifices (e.g., having circular, square, or other shapes) to form a plurality of extruded bodies. These extruded bodies may be allowed to recombine such as via gravity to form an extruded body having channels. In yet another embodiment, channels may be formed into the structured composition such as by lightly embossing or stamping the channels therein. In methods including the step of imparting a shape to the composition, such step may occur prior to (sequentially), or simultaneously with the contacting step.

The methods of the present invention preferably comprise the use of a composition suitable for having a shape imparted thereon or thereto, and tending to retain such shape, so as to form a plurality of channels upon application to a substrate, such as a structured composition. As used herein the term “structured composition,” means a composition having a Yield Stress from about 1 Pascal (Pa) to about 1500 Pa as measured via the “Yield Stress Test” described in the Test Methods below. Examples of certain preferred structured compositions include those having a Yield Stress of from about 1 Pa to about 1500 Pa, preferably from about 10 Pa to about 1100 Pa, as measured by the Yield Stress Test. In certain embodiments, the composition preferably includes one or more of lamellar, spherulitic, liquid-crystal and/or optically birefringent phases phase that is largely composed of one or more surfactants that is dispersed within an exterior (typically aqueous) phase. The viscosity of the personal care composition may be such that the composition is spreadable such as that of a cream or lotion or gel. For example, when measured using a LVT3 spindle at 30 rpm, the viscosity may be from about 500 cps to about 2000 cps.

Any suitable structured compositions disclosed herein or known in the art may be used in accord with the present method. The structured compositions suitable for the present invention tend to be beneficial in that they can have a shape or shapes imparted thereto, including a shape suitable for defining channels when applied to skin in accord with present invention. In certain embodiments, the structured composition of the present invention “remembers” the shape imparted thereto for a period of time. This period of time, i.e., “memory period” or “relaxation time” is generally at least about 2 seconds, preferably at least about 5 seconds. In certain embodiments, the relaxation time is at least about 1 minute, and, in other preferred embodiments, at least about 3 minutes.

To help provide suitable memory to the composition, the composition may include one or more of various surfactants; e.g., anionic, nonionic, cationic, and amphoteric surfactants. While the amount of total surfactant in the composition is variable, for certain end-use application such as body washes, shampoos, facial cleansers and the like, it is desirable to have a total surfactant concentration that is at least about 10%, such as from about 10% to about 90%, preferably from about 20% to about 70%, more preferably from about 30% to about 60%.

Any of a variety of suitable anionic surfactants may be used in the present invention. According to certain embodiments, suitable anionic surfactants may be branched or unbranched and may include alkyl sulfates, alkyl ether sulfates, alkyl monoglyceryl ether sulfates, alkyl sulfonates, alkylaryl sulfonates, alkyl sulfosuccinates, alkyl ether sulfosuccinates, alkyl sulfosuccinamates, alkyl amidosulfosuccinates, alkyl carboxylates, alkyl amidoethercarboxylates, alkyl succinates, fatty acyl sarcosinates, fatty acyl amino acids, fatty acyl taurates, fatty alkyl sulfoacetates, alkyl phosphates, and mixtures of two or more thereof. Examples of certain anionic surfactants include:

alkyl sulfates of the formula

R′—CH₂OSO₃X′;

alkyl ether sulfates of the formula

R′(OCH₂CH₂)ᵥOSO₃X′;

alkyl monoglyceryl ether sulfates of the formula

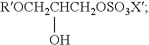

alkyl monoglyceride sulfates of the formula

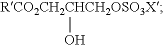

alkyl monoglyceride sulfonates of the formula

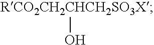

alkyl sulfonates of the formula

R′—SO₃X′;

alkylaryl sulfonates of the formula

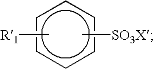

alkyl sulfosuccinates of the formula:

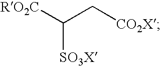

alkyl ether sulfosuccinates of the formula:

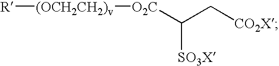

alkyl sulfosuccinamates of the formula:

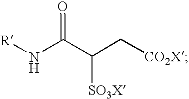

alkyl amidosulfosuccinates of the formula

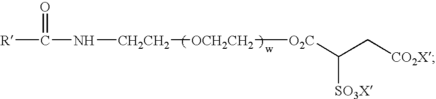

alkyl carboxylates of the formula:

R′—(OCH₂CH₂)_{w}—OCH₂CO₂X′;

alkyl amidoethercarboxylates of the formula:

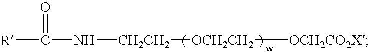

alkyl succinates of the formula:

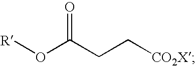

fatty acyl sarcosinates of the formula:

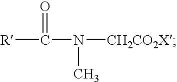

fatty acyl amino acids of the formula:

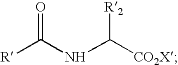

fatty acyl taurates of the formula:

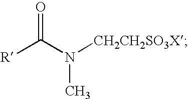

fatty alkyl sulfoacetates of the formula:

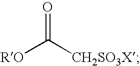

alkyl phosphates of the formula:

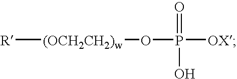

wherein
R′ is an alkyl group having from about 7 to about 22, and preferably from about 7 to about 16 carbon atoms,
R′₁ is an alkyl group having from about 1 to about 18, and preferably from about 8 to about 14 carbon atoms,
R′₂ is a substituent of a natural or synthetic I-amino acid,
X′ is selected from the group consisting of alkali metal ions, alkaline earth metal ions, ammonium ions, and ammonium ions substituted with from about 1 to about 3 substituents, each of the substituents may be the same or different and are selected from the group consisting of alkyl groups having from 1 to 4 carbon atoms and hydroxyalkyl groups having from about 2 to about 4 carbon atoms and
v is an integer from 1 to 6;
w is an integer from 0 to 20;
   and mixtures thereof.

In certain preferred embodiments, the anionic surfactant for use in the present invention comprises a branched anionic surfactant. By “branched anionic surfactant,” it is meant an anionic surfactant comprising more than 10% branched surfactant molecules. Suitable branched anionic surfactants include tridecanol based sulfates such as sodium trideceth sulfate, which generally comprises a high level of branching, with over 80% of surfactant molecules comprising at least 2 branches. Another suitable branched anionic surfactant is a C₁₂₋₁₃ alkyl sulfate derived from SAFOL 23 alcohol (Sasol, Inc, Houston, Tex., USA) which has about 15-30% branched surfactant molecules.

Branched anionic surfactants include but are not limited to the following branched anionic alkyl sulfate or alkyl ether sulfate surfactants: sodium tridecyl sulfate, sodium C₁₂₋₁₃ alkyl sulfate, sodium C₁₂₋₁₅ alkyl sulfate, sodium C₁₂₋₁₅ alkyl sulfate, sodium C₁₂₋₁₈ alkyl sulfate, sodium C₁₀₋₁₆ alkyl sulfate, sodium trideceth sulfate, sodium C₁₂₋₁₃ pareth sulfate, sodium C₁₂₋₁₃ pareth-n sulfate, and sodium C₁₂₋₁₄ pareth-n sulfate. One particularly suitable branched anionic surfactant (about 50% branched) is a sodium trideceth sulfate, available as CEDEPAL TD 430 MFLD from Stepan Company of Northfield, Ill.

Other salts of all the aforementioned branched anionic surfactants are useful, such as TEA, DEA, ammonia, potassium salts. Useful alkoxylates include the ethylene oxide, propylene oxide and EO/PO mixed alkoxylates. Phosphates, carboxylates and sulfonates prepared from branched alcohols are also useful anionic branched surfactants. Branched anonic surfactants can be derived from synthetic alcohols such as the primary alcohols from the liquid hydrocarbons produced by Fischer-Tropsch condensed syngas, for example SAFOL 23 Alcohol available from Sasol North America, Houston, Tex.; from synthetically made alcohols such as those described in U.S. Pat. No. 6,335,312 issued to Coffindaffer, et al on Jan. 1, 2002. Preferred alcohols are SAFOL. 23. Preferred alkoxylated alcohols are SAFOL 23-3. Sulfates can be prepared by conventional processes to high purity from a sulfur based SO₃ air stream process, chlorosulfonic acid process, sulfuric acid process, or Oleum process. Preparation via SO₃ air stream in a falling film reactor is a preferred sulfation process.

Suitable branched anionic surfactants include but are not limited to the branched anionic sulfates derived from SAFOL 23-n as previously described, where n is an integer between 1 and about 20. Fractional alkloxylation is also useful, for example by stoichiometrically adding only about 0.3 moles EO, or 1.5 moles EO, or 2.2 moles EO, based on the moles of alcohol present, since the molecular combinations that result are in fact always distributions of alkoxylates so that representation of n as an integer is merely an average representation. Preferred monomethyl branched anionic surfactants include a C₁₂₋₁₃ alkyl sulfate derived from the sulfation of SAFOL 23, which has about 28% branched anionic surfactant molecules.

When the branched anionic surfactant is a branched anionic primary sulfate, it may contain some of the following branched anionic surfactant molecules: 4-methyl undecyl sulfate, 5-methyl undecyl sulfate, 7-methyl undecyl sulfate, 8-methyl undecyl sulfate, 7-methyl dodecyl sulfate, 8-methyl-dodecyl sulfate, 9-methyl dodecyl sulfate,4,5-dimethyl decyl sulfate, 6,9-dimethyl decyl sulfate, 6,9-dimethyl undecyl sulfate, 5-methyl-8-ethyl undecyl sulfate, 9-methyl undecyl sulfate, 5,6,8-trimethyl decyl sulfate, 2-methyl dodecyl sulfate, and 2-methyl undecyl sulfate. When the anionic surfactant is a primary alkoxylated sulfate, these same molecules may be present as the n=0 unreacted alcohol sulfates, in addition to the typical alkoxylated adducts that result from alkoxylation.

Any amounts of anionic surfactant or combinations thereof suitable to, in conjunction with other ingredients in the composition to produce a structured composition is suitable. According to certain embodiments, branched anionic surfactant is used in a concentration from greater than about 0.1% to about 20% by weight of active branched anionic surfactant in the composition. In certain embodiments, the branched anionic surfactant is in present in a concentration from about 0.3% to about 15%, more preferably from about 2% to about 13%, even more preferably from about 4.5% to about 10% of active branched anionic surfactant in the composition.

Additional surfactants, such as amphoteric, cationic, non-ionic, or combinations thereof may be used in compositions of the present invention. For example, any of a variety of amphoteric surfactants are suitable for use in the present invention. The amphoteric surfactants are disclosed herein without a counter ion. One skilled in the art would readily recognize that under the pH conditions of the compositions of the present invention, the amphoteric surfactants are either electrically neutral by virtue of having balancing positive and negative charges, or they have counter ions such as alkali metal, alkaline earth, or ammonium counter ions.

Examples of amphoteric surfactants include, but are not limited to “betaines.” Any of a variety of suitable betaines may be used in the compositions of the present invention. Examples of suitable betaines include alkyl betaines; amidoalkyl betaines; amidoalkyl sultaines; amphophosphates; phosphorylated imidazolines such as phosphobetaines and pyrophosphobetaines, as well as other betaines represented by the following formula:

B—N⁺R₁R₂(CH₂)ₚX⁻

wherein B is an alkyl or alkenyl group, preferably a group having from about 7 to about 22 carbon atoms; and X⁻ is a anionically charged moiety or a neutral (protonated) derivative thereof. As will be recognized by those of skill in the art, the charge on X⁻ may be dependent on the pH of the composition.

Examples of suitable alkyl betaines include those compounds of the formula:

D-N⁺R₉R₁₀(CH₂)ₚCO₂⁻

wherein
D is an alkyl or alkenyl group having from about 8 to about 22, e.g., from about 8 to about 16 carbon atoms;
R₉ and R₁₀ are each independently an alkyl or hydroxyalkyl group having from about 1 to about 4 carbon atoms; and
p is 1 or 2.
   A preferred betaine for use in the present invention is lauryl betaine, available commercially from Huntsman International LLC of The Woodlands, Tex., as “Empigen BB/J.”

Examples of suitable amidoalkyl betaines include those compounds of the formula:

F—CO—NH(CH₂)_{q}—N⁺R₁₁R₁₂(CH₂)ₘCO₂⁻
wherein
   F is an alkyl or alkenyl group having from about 7 to about 21, e.g. from about 7 to about 15 carbon atoms;
R₁₁ and R₁₂ are each independently an alkyl or Hydroxyalkyl group having from about 1 to about 4 carbon atoms;

q is an integer from about 2 to about 6; and m is 1 or 2.

One amidoalkyl betaine is cocamidopropyl betaine, available commercially from Degussa Goldschmidt Chemical Corporation of Hopewell, Va. under the tradename, “Tegobetaine L7.”

Examples of suitable amidoalkyl sultaines include those compounds of the formula

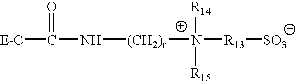

wherein
   E is an alkyl or alkenyl group having from about 7 to about 21, e.g. from about 7 to about 15 carbon atoms;
   R₁₄ and R₁₅ are each independently an alkyl, or hydroxyalkyl group having from about 1 to about 4 carbon atoms;
   r is an integer from about 2 to about 6; and
   R₁₃ is an alkylene or hydroxyalkylene group having from about 2 to about 3 carbon atoms;

In one embodiment, the amidoalkyl sultaine is cocamidopropyl hydroxysultaine, available commercially from Rhodia Inc. of Cranbury, N.J. under the tradename, “Mirataine CBS.”

Examples of suitable amphophosphates compounds include those of the formula:

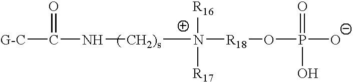

wherein
   G is an alkyl or alkenyl group having about 7 to about 21, e.g. from about 7 to about 15 carbon atoms;
      s is an integer from about 2 to about 6;
      R₁₆ is hydrogen or a carboxyalkyl group containing from about 2 to about 3 carbon atoms;
      R₁₇ is a hydroxyalkyl group containing from about 2 to about 3 carbon atoms or a group of the formula:

R₁₉—O—(CH₂)ₜ—CO₂⁻
wherein
R₁₉ is an alkylene or hydroxyalkylene group having from about 2 to about 3 carbon atoms and
t is 1 or 2; and
R₁₈ is an alkylene or hydroxyalkylene group having from about 2 to about 3 carbon atoms.

In one embodiment, the amphophosphate compounds are sodium lauroampho PG-acetate phosphate, available commercially from Uniqema of Chicago, Ill. under the tradename, “Monateric 1023,” and those disclosed in U.S. Pat. No. 4,380,637, which is incorporated herein by reference.

Examples of suitable phosphobetaines include those compounds of the formula:

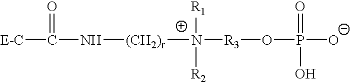

wherein E, r, R₁, R₂ and R₃, are as defined above. In one embodiment, the phosphobetaine compounds are those disclosed in U.S. Pat. Nos. 4,215,064, 4,617,414, and 4,233,192, which are all incorporated herein by reference.

Examples of suitable pyrophosphobetaines include those compounds of the formula:

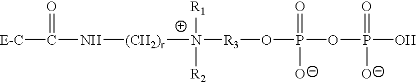

wherein E, r, R_{1, R}₂ and R₃, are as defined above. In one embodiment, the pyrophosphobetaine compounds are those disclosed in U.S. Pat. Nos. 4,382,036, 4,372,869, and 4,617,414, which are all incorporated herein by reference.

Any amount of betaine or combination of betaines suitable, in conjunction with other ingredients in the composition, to produce a structured composition may be used in accord with the invention. According to certain embodiments, betaine is used in a concentration from greater than about 0.1% to about 50% by weight of active betaine in the composition. Preferably, betaine is in present in a concentration from about 1% to about 40%, more preferably from about 5% to about 40%, even more preferably from about 15% to about 35% of active betaine in the composition.

Other suitable amphoteric surfactants include amphocarboxylates such as alkylamphoacetates (mono or di); phosphorylated imidazolines such as phosphobetaines and pyrophosphobetaines; carboxyalkyl alkyl polyamines; alkylimino-dipropionates; alkylamphoglycinates (mono or di); alkylamphoproprionates (mono or di),); N-alkyl β-aminoproprionic acids; alkylpolyamino carboxylates; and mixtures thereof.

Examples of suitable amphocarboxylate compounds include those of the formula:

A-CONH(CH₂)ₓN⁺R₅R₆ R₇
wherein
A is an alkyl or alkenyl group having from about 7 to about 21, e.g. from about 10 to about 16 carbon atoms;
x is an integer of from about 2 to about 6;
R₅ is hydrogen or a carboxyalkyl group containing from about 2 to about 3 carbon atoms;
R₆ is a hydroxyalkyl group containing from about 2 to about 3 carbon atoms or is a group of the formula:

R₈—O—(CH₂)ₙCO₂⁻
wherein
R₈ is an alkylene group having from about 2 to about 3 carbon atoms and n is 1 or 2; and
R₇ is a carboxyalkyl group containing from about 2 to about 3 carbon atoms;

Examples of suitable amphophosphate compounds include those of the formula:

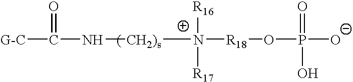

wherein
   G is an alkyl or alkenyl group having about 7 to about 21, e.g. from about 7 to about 15 carbon atoms;
      s is an integer from about 2 to about 6;
      R₁₆ is hydrogen or a carboxyalkyl group containing from about 2 to about 3 carbon atoms;
      R₁₇ is a hydroxyalkyl group containing from about 2 to about 3 carbon atoms or a group of the formula:

R₁₉—O—(CH₂)ₜ—CO₂⁻
wherein
R₁₉ is an alkylene or hydroxyalkylene group having from about 2 to about 3 carbon atoms and
t is 1 or 2; and
R₁₈ is an alkylene or hydroxyalkylene group having from about 2 to about 3 carbon atoms.

In one embodiment, the amphophosphate compounds are sodium lauroampho PG-acetate phosphate, available commercially from Uniqema of Chicago, Ill. under the tradename, “Monateric 1023,” and those disclosed in U.S. Pat. No. 4,380,637, which is incorporated herein by reference.

Examples of suitable carboxyalkyl alkylpolyamines include those of the formula:

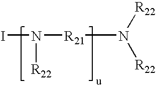

wherein
   1 is an alkyl or alkenyl group containing from about 8 to about 22, e.g. from about 8 to about 16 carbon atoms;
   R₂₂ is a carboxyalkyl group having from about 2 to about 3 carbon atoms;
   R₂₁ is an alkylene group having from about 2 to about 3 carbon atoms and
   u is an integer from about 1 to about 4.

In one embodiment, in order to provide a high degree of cost-effectiveness, the weight fraction of betaine relative to all amphoteric surfactants in the composition is at least about 25%, preferably at least about 50%, and most preferably at least about 75%.

Various nonionic surfactants may also be suitable. Examples of suitable nonionic surfactants include, but are not limited to, fatty alcohol acid or amide ethoxylates, monoglyceride ethoxylates, sorbitan ester ethoxylates alkyl polyglycosides, mixtures thereof, and the like. Certain preferred nonionic surfactants include polyoxyethylene derivatives of polyol esters, wherein the polyoxyethylene derivative of polyol ester (1) is derived from (a) a fatty acid containing from about 8 to about 22, and preferably from about 10 to about 14 carbon atoms, and (b) a polyol selected from sorbitol, sorbitan, glucose, α-methyl glucoside, polyglucose having an average of about 1 to about 3 glucose residues per molecule, glycerine, pentaerythritol and mixtures thereof, (2) contains an average of from about 10 to about 120, and preferably about 20 to about 80 oxyethylene units; and (3) has an average of about 1 to about 3 fatty acid residues per mole of polyoxyethylene derivative of polyol ester. Examples of such preferred polyoxyethylene derivatives of polyol esters include, but are not limited to PEG-80 sorbitan laurate and Polysorbate 20. PEG-80 sorbitan laurate, which is a sorbitan monoester of lauric acid ethoxylated with an average of about 80 moles of ethylene oxide, is available commercially from Uniqema of Chicago, Ill. under the tradename, “Atlas G-4280.” Polysorbate 20, which is the laurate monoester of a mixture of sorbitol and sorbitol anhydrides condensed with approximately 20 moles of ethylene oxide, is available commercially from ICI Surfactants of Wilmington, Del. under the tradename “Tween 20.”

Another class of suitable nonionic surfactants includes long chain alkyl glucosides or polyglucosides, which are the condensation products of (a) a long chain alcohol containing from about 6 to about 22, and preferably from about 8 to about 14 carbon atoms, with (b) glucose or a glucose-containing polymer. Preferred alkyl gluocosides comprise from about 1 to about 6 glucose residues per molecule of alkyl glucoside. A preferred glucoside is decyl glucoside, which is the condensation product of decyl alcohol with a glucose polymer and is available commercially from Cognis Corporation of Ambler, Pa. under the tradename, “Plantaren 2000.”

Any amounts of nonionic surfactant suitable to produce a structured composition may be combined according to the present methods. For example, the amount of monomeric surfactants used in the present invention may be from about 2% to about 30%, more preferably from about 3% to about 25%, even more preferably from about 8% to about 20% of total active nonionic surfactant in the composition, and even more preferably from about 9% to about 15%.

Various cationic surfactants may also be suitable for use in the present compositions. Examples of suitable cationic surfactants include, but are not limited to alkyl quaternaries (mono, di, or tri), benzyl quaternaries, ester quaternaries, ethoxylated quaternaries, alkyl amines, and mixtures thereof, wherein the alkyl group has from about 6 carbon atoms to about 30 carbon atoms, with about 8 to about 22 carbon atoms being preferred.

In order to provide suitable memory to the composition, the composition, in certain preferred embodiments includes a structuring agent. By “structuring agent” it is meant, a molecule that, particularly when combined with various surfactants, is capable of rendering the composition into any of various “structured phases” such as lamellar, spherulitic, liquid-crystal and/or optically birefringent phases, etc. Suitable structuring agents include those known in the art such as unsaturated and/or branched long chain (C₈-C₂₄) liquid fatty compounds such as fatty acids fatty esters and fatty alcohols; short chain saturated fatty acids such as capric acid or caprylic acid, among other structurants.

In one embodiment, the structuring agents is a branched fatty alcohol. Any of a variety of branched fatty alcohols may be used in the present compositions. By “branched fatty alcohol”, it is meant, any of various alcohols derived from plant or animal oils and fats having at least one pendant hydrocarbon-comprising chain. The branched fatty alcohol may comprise any number of carbon atoms, preferably from about 7 to about 22 carbon atoms, more preferably about 9 to about 15 carbon atoms, and even more preferably about 11 to about 15 carbon atoms. Suitable branched fatty alcohols may comprise one or more alcohol groups per molecule. In certain preferred embodiments, the fatty alcohol comprises one alcohol group per molecule.

In one embodiment, the branched fatty alcohols comprise one or more branches in the carbon backbone of the molecule. In certain preferred embodiments, the branched fatty alcohol is monobranched. By “monobranched”, it is meant the fatty alcohol has an alkyl chain with one (CH) functional group resulting in one branch in the alkyl chain, i.e. the fatty alcohol has one and only one carbon that has one hydrogen atom and three carbon atoms bonded thereto.

In certain preferred embodiments, the branched fatty alcohol is a primary alcohol. By “primary alcohol,” it is meant no —COH group is bonded to more than one carbon atom.

In one particularly preferred embodiment, the branched fatty alcohol is both monobranched and a primary alcohol. In a more particularly preferred embodiment, the branched fatty alcohol is both monobranched and a primary alcohol and has only one alcohol group per molecule.

In certain preferred embodiments, the branched fatty alcohol consists solely of hydrogen, carbon, and oxygen atoms. The carbon-carbon bonds within the branched fatty alcohol may be saturated or unsaturated.

In one particularly preferred embodiment, the branched fatty alcohol is a monobranched primary fatty alcohol that can be represented by the following structure:

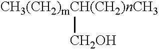

wherein each of the three following equations are satisfied: m+n=8 or 9; and m is an integer that ranges from 0 to 9 (inclusive); and n is an integer that ranges from 0 to 9 (inclusive).

Commercially available materials that are particularly suitable for use as the branched fatty alcohol include the following materials alone or in combination: Isalchem 123 or Lialchem 123 produced by Sasol Chemical Co of Bad Homburg, Germany. In a particularly preferred embodiment, the branched fatty alcohol is Isalchem 123.

In another embodiment, the branched fatty alcohol includes an alkoxylate moiety, such as ethoxy and/or propoxy groups. Any number of alkoxy groups are acceptable as long as the fatty alcohol is still capable of providing a structured composition. In one embodiment, the fatty alcohol has up to an including 10 alkoxy groups, more preferably from 0 to 3 alkoxy groups, most preferably from 1 to 3 alkoxy groups.

The concentration of the branched fatty alcohol in the composition of the invention is preferably from about 0.1% to about 10% by weight of active branched fatty alcohol in the composition, more preferably from 0.5% to about 5% by weight, even more preferably from about 0.75% to about 4%. In one embodiment of the invention, the branched fatty alcohol and betaine are present in a fatty alcohol to betaine (weight to weight, on an actives basis) ratio that is from about 0.15:1 to about 0.35:1.

As will be recognized by those of skill in the art, the compositions of the present invention further comprise water, which serves to provide a vehicle about which a structured phase is dispersed. The concentration of water in the composition is sufficient to stabilize the composition, but not so great as to prevent the composition from becoming structured. In one embodiment, the concentration of water is from about 5% to about 70%, preferably from about 15% to about 60%, more preferably from about 20% to about 50%, and most preferably from about 25% to about 45%.

In certain embodiments of the invention, compositions of the present invention include other functional ingredients. By other functional ingredients it is meant any moiety that serves one or more functions either to stabilize or provide aesthetic benefits to the composition or to impart one or more of various benefits to the end user. These various functional ingredients may be of any form at room temperature (e.g., solids, liquids, pastes and the like) and be dispersed, emulsified, or solubilized or otherwise homogenized within the composition.

A wide variety of functional ingredients may be used in compositions of the present invention, although it is preferred that the ingredient does not adversely affect the phase stability of the composition. By “adversely effect the phase stability,” it is meant that by including the particular functional ingredient, when subject to a stability challenge (e.g., held at 22° C., 50% relative humidity for a week; when subject to three 48 hour freeze-thaw cycles) the composition irrevocably phase separates into two or more visually distinct phases so as to be displeasing (e.g., in a tactile, olfactory, and/or visual sense) for topical use.

Functional ingredients that may be used include, but are in no way limited to: dyes and colorants; ultraviolet filters and suncsreens, opacificiers, matting agents, rheology modifiers, oils, emollients, and skin conditioners; chelating and sequestering agents, pH adjusters, humectants, film forming polymers, plasticizers, fragrance components; water soluble solvents such as glycols including glycerol, propylene glycol C1-C6 alcohols may be incorporated into the composition (again, as long as There is no adverse effect on phase stability) and various benefit agents, as described below.

The functional ingredient may be water-insoluble. By “water-insoluble,” it is meant, a moiety that cannot be rendered essentially completely soluble in deionized water at 25° C., after providing a 1% by weight of said moiety in said deionized water under moderate agitation for 10 minutes. A wide variety of water-insoluble components may be incorporated into compositions of the present invention. The structured nature of the composition is suitable for dispersing water insoluble components that are solid at room temperature (e.g., certain polymers and waxes; dyes; and particulates such as mineral oxides, silicates, aluminosilicates, zinc pyrithione, colloidal oat flour, soy derivatives and the like) or liquid at room temperature (e.g., oils, emollients, and skin conditioners; biological actives; fragrance components).

By way of example, any of a variety of commercially available pearlescent or opacifying agents which are capable of suspending water insoluble additives such as silicones and/or which tend to indicate to consumers that the resultant product is a conditioning shampoo are suitable for use in this invention. The pearlescent or opacifying agent may be present in an amount, based upon the total weight of the composition, of from about 1 percent to about 10 percent, e.g. from about 1.5 percent to about 7 percent or from about 2 percent to about 5 percent. Examples of suitable pearlescent or opacifying agents include, but are not limited to mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)ₐ—H, wherein J is an alkylene group having from about 2 to about 3 carbon atoms; and a is 2 or 3;fatty alcohols containing from about 16 to about 22 carbon atoms; fatty esters of the formula: KCOOCH₂L, wherein K and L independently contain from about 15 to about 21 carbon atoms; inorganic solids insoluble in the shampoo composition, and mixtures thereof

The pearlescent or opacifying agent may be introduced to the structured composition as a pre-formed, stabilized aqueous dispersion, such as that commercially available from Cognis Corporation of Ambler, Pa. under the tradename, “Euperlan PK-3000.” This material is a combination of glycol distearate (the diester of ethylene glycol and stearic acid), Laureth-4 (CH₃(CH₂)₁₀CH₂(OCH₂CH₂)₄OH) and cocamidopropyl betaine and may be in a weight percent ratio of from about 25 to about 30: about 3 to about 15: about 20 to about 25, respectively.

Any of a variety of commercially available secondary conditioners, such as volatile silicones, which impart additional attributes, such as gloss to the hair are suitable for use in this invention. The volatile silicone conditioning agent has an atmospheric pressure boiling point less than about 220° C. The volatile silicone conditioner may be present in an amount of from about 0 percent to about 3 percent, e.g. from about 0.25 percent to about 2.5 percent or from about 0.5 percent to about 1.0 percent, based on the overall weight of the composition. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Mich. under the tradename, “DC-345” and mixtures thereof, and preferably include cyclomethicone fluids. Other suitable secondary conditioners include cationic polymers, including polyquarterniums, cationic guar, and the like.

Any of a variety of commercially available humectants, which are capable of providing moisturization and conditioning properties to the personal cleansing composition, are suitable for use in the present invention. The humectant may be present in an amount of from about 0 percent to about 10 percent, e.g. from about 0.5 percent to about 5 percent or from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, dipropylene glycol, polyglycerols, and mixtures thereof; 2) polyalkylene glycol of the formula: HO—(R″O)_{b}—H, wherein R″ is an alkylene group having from about 2 to about 3 carbon atoms and b is an integer of from about 2 to about 10; 3) polyethylene glycol ether of methyl glucose of formula CH₃—C₆H₁₀O₅—(OCH₂CH₂)_{c}—OH, wherein c is an integer from about 5 to about 25; 4) urea; and 5) mixtures thereof, with glycerine being the preferred humectant.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the tradename, “Versene 100XL” and is present in an amount, based upon the total weight of the composition, from about 0 to about 0.5 percent or from about 0.05 percent to about 0.25 percent.

Suitable preservatives include, for example, parabens, quaternary ammonium species, phenoxyethanol, benzoates, DMDM hydantoin, and are present in the composition in an amount, based upon the total weight of the composition, from about 0 to about 1 percent or from about 0.05 percent to about 0.5 percent.

While it is typically unnecessary to include thickening agents in the composition (since the “thickening” is typically aesthetically and cost-effectively accomplished using the combination of betaine and the fatty alcohol), it is possible to incorporate any of a variety of commercially available thickening agents, which are capable of imparting the appropriate viscosity to the personal cleansing compositions are suitable for use in this invention.

Examples of suitable thickening agents nonexclusively include: mono or diesters of 1) polyethylene glycol of formula: HO—(CH₂CH₂O)_{z}H, wherein z is an integer from about 3 to about 200; and 2) fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; hydrophobically-modified alkali swellable emulsions (HASEs); hydrophobically-modified ethoxylated urethanes (HEURs); xantham and guar gums; and mixtures thereof. Preferred thickeners include polyethylene glycol ester, and more preferably PEG-150 distearate which is available from the Stepan Company of Northfield, Ill. or from Comiel, S.p.A. of Bologna, Italy under the tradename, “PEG 6000 DS”.

Compositions of the present invention may include a benefit agent. A benefit agent is any element, an ion, a compound (e.g., a synthetic compound or a compound isolated from a natural source) or other chemical moiety in solid (e.g. particulate), liquid, or gaseous state and compound that has a cosmetic or therapeutic effect on the skin, hair, mucosa, or teeth. As used herein, the term “benefit agent” includes any active ingredient such as a cosmetic or pharmaceutical, that is to be delivered into and/or onto the skin, hair, mucosa, or teeth at a desired location.

The benefit agents useful herein may be categorized by their therapeutic benefit or their postulated mode of action. However, it is to be understood that the benefit agents useful herein may, in some circumstances, provide more than one therapeutic benefit or operate via greater than one mode of action. Therefore, the particular classifications provided herein are made for the sake of convenience and are not intended to limit the benefit agents to the particular application(s) listed.

Examples of suitable benefit agents include those that provide benefits such as, but not limited to: emollients, moisturizing and water-loss prevention agents; cleansing agents; depigmentation agents; reflectants and optical modifiers; amino acids and their derivatives; antimicrobial agents; allergy inhibitors; anti-acne agents; anti-aging agents; anti-wrinkling agents, antiseptics; analgesics; shine-control agents; antipruritics; local anesthetics; anti-hair loss agents; hair growth promoting agents; hair growth inhibitor agents, antihistamines; antiinfectives; anti-inflammatory agents; anticholinergics; vasoconstrictors; vasodilators; wound healing promoters; peptides, polypeptides and proteins; deodorants and antiperspirants; medicament agents; skin firming agents, vitamins; skin lightening agents; skin darkening agents; antifungals; depilating agents; counterirritants; hemorrhoidals; insecticides; enzymes for exfoliation or other functional benefits; enzyme inhibitors; poison ivy products; poison oak products; burn products; anti-diaper rash agents; prickly heat agents; vitamins; herbal extracts; vitamin A and its derivatives; flavenoids; sensates and stress-reducing agents; anti-oxidants; hair lighteners; sunscreens; anti-edema agents, neo-collagen enhancers, anti-dandruff/sebhorreic dermatitis/psoriasis agents; keratolytics; lubricants; lightening and whitening agents; calcification, fluoridation and mineralization agents; and mixtures thereof.

The amount of the benefit agent that may be used may vary depending upon, for example, the ability of the benefit agent to penetrate through the skin, nail, mucosa, or teeth; the specific benefit agent chosen, the particular benefit desired, the sensitivity of the user to the benefit agent, the health condition, age, and skin and/or nail condition of the user, and the like. In sum, the benefit agent is used in a “safe and effective amount,” which is an amount that is high enough to deliver a desired skin or nail benefit or to modify a certain condition to be treated, but is low enough to avoid serious side effects, at a reasonable risk to benefit ratio within the scope of sound medical judgment.

Compositions of the present invention are structured, i.e., have a Yield Stress from about 1 Pascal (Pa) to about 1500 Pa; more preferably from about 10 Pa to about 1100 Pa and preferably include a lamellar phase that is largely composed of one or more surfactants that is dispersed within an exterior (typically aqueous) phase. The viscosity of the personal care composition may be such that the composition is spreadable such as that of a cream or lotion or gel. For example. when measured using a LVT3 spindle at 30 rpm, the viscosity may be from about 500 cps to about 2000 cps.

The pH of the present compositions is not critical, but may be in a range that does not facilitate irritation to the skin, such as from about 5 to about 7.5.

In one embodiment of the present invention the structured composition comprises at least two visually distinct phases wherein a first phase is visually distinct from a second phase. Preferably, the visually distinct phases are packaged in physical contact with one another and are stable. Preferably, the visually distinct phases form a pattern such as stripes, ribbons, or striations. The ratio of a first phase to a second phase is typically from about 1:99 to about 99:1, preferably from 90:10 to about 10:90, more preferably about from 70:30 to about 30:70, still even more preferably about 50:50. As known in the art, the first visually distinct phase may include the components in a manner sufficient to provide structure, e,g, betaine, branched anionic surfactant, and branched fatty alcohol. The second visually distinct phase may also include the above-mentioned components in a manner sufficient to provide structure. Alternatively, the second phase may be unstructured.

The applicants have further discovered that the reduction of wet slip may be reduced by employing a container suitable to be held in the hand of a user, the container including an orifice that defines a shape that includes a plurality of protrusions and a plurality of indentations. Within the container is a structured composition. When the structured composition extruded through the orifice, the structured composition is suitable to conform sufficiently to the shape of the orifice, such that an extruded portion of the structured composition comprises a plurality of contact surfaces for contacting a body surface and a plurality of channels intermediate the contact surfaces.

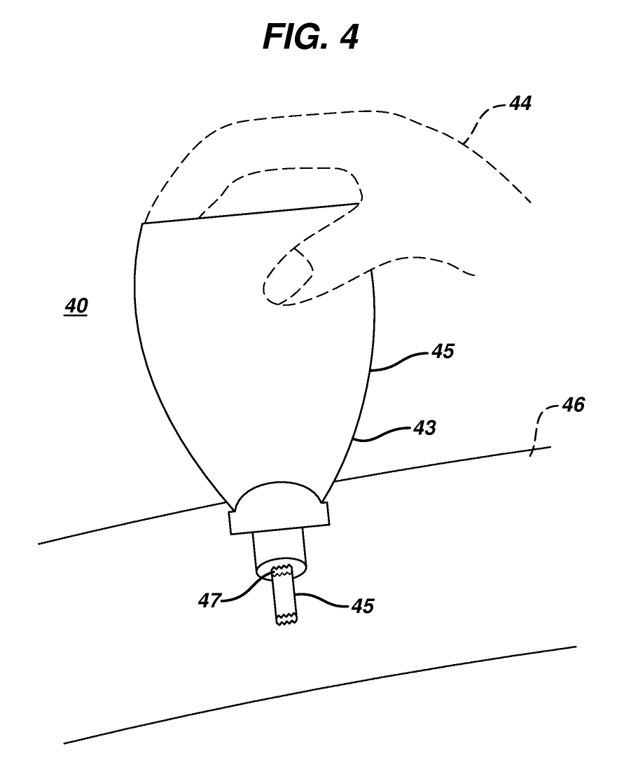

FIG. 4 is a perspective view of a personal care product 40 that is consistent with embodiments of the invention described herein. The personal care product 40 includes a container 43 and a composition within the container 43. The container further includes one or more walls 45 for containing the composition and an orifice 47 for expressing the composition from the container 43 onto a substrate 46.

While the container 43 may be of varying size, shape, and relative dimensions, the container 43 is generally suitable to be held in a user's hand 44. The dimensions of the container 43 may be such that the one or more walls 45 can readily be squeezed, such as between a thumb and one or more fingers, as shown in FIG. 4, in order to induce portions of the composition within to move outside the container. The container may enclose a volume of, for example, from about 1 ml to about 1000 ml within which the composition is housed.

While FIG. 4 depicts container 43 being squeezed to express the composition therefrom, other means of expressing the composition are contemplated, e.g., such as by merely inverting the container and allowing gravity to express the composition; by pumping via, for example, pushing, twisting or otherwise actuating a hand pump; among other methods for commonly expressing viscous personal care formulations from containers.

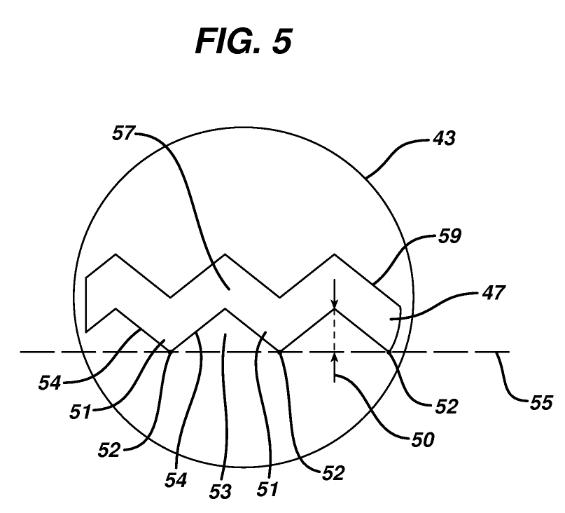

FIG. 5 depicts a top view of the orifice 47 of container 43. The orifice 47 has a boundary 59 within a plane (shown in FIG. 5 as the plane of the paper). The area defined by the boundary 59 is not critical, and may selected based upon the factors such as the particular composition, the bioactivity of the composition, the substrate 46 to which it is being applied, etc. In one embodiment, the area of the boundary 59 is from about 0.005 cm² to about 20 cm². In one preferred embodiment, the boundary 59 of the orifice 47 useful for a body wash or shampoo composition is from about 0.04 cm² to about 0.5 cm². In one preferred embodiment the boundary 59 of the orifice 47 useful for a topical moisturizer or skin cream composition is from about 0.05 cm² to about 0.2 cm². In one preferred embodiment, the boundary 59 of the orifice 47 useful for an eye medication is from about 0.005 cm² to about 0.04 cm².

The boundary 59 defines a shape that includes a plurality of protrusions 51 and a plurality of indentations 53. By the terms “protrusions” and “indentations” it is meant the following: when considering the orifice 47 and orienting it such that the shape of the orifice is in its lowest energy position (such that its center of gravity 57 is the lowest), one can draw an imaginary line 55 that just contacts one or more terminal points 52 on the boundary 59 from underneath, yet does not cross the boundary 59. Each of the terminal points 52 uniquely identify a protrusion. The imaginary line 55 thus constructed itself forms, in the plane of the paper, an area fully enclosed by the imaginary line 55 and at least portions of the boundary 59. Two indentations and three protrusions are thus defined in FIG. 5.

In general, the protrusions and indentations defined by the orifice may be substantially of the same or different sizes and shapes. In certain preferred embodiments, the orifice defines a boundary which comprises protrusions and indentations of substantially the same size and shape as shown in FIG. 5.

The boundary 59 encloses an area that generally determines the cross-sectional area of the extruded portion and thus the volume of the extruded portion per unit length that is extruded. Thus for compositions of similar density, a larger area of the orifice implies a larger mass of extruded composition per until length. A larger mass of extruded composition may encourage greater slip due to the force of gravity.

The area of the boundary of the orifice may be varied depending upon how much composition it is desirable to use at a given time. In one embodiment of the area defined by the boundary is from about 0.05 cm² to about 0.5 cm², such as from about 0.1 cm² to about 0.4 cm², such as from about 0.15 cm² to about 0.4 cm², such as from about 0.2 cm² to about 0.4 cm²

The number of protrusions and indentations are not critical, but the inventors have found that at least two indentations are required. In one embodiment, the boundary of the orifice 7 has from 2 to about 100 indentations, more preferably from about 2 to about 20 indentations, even more preferably from about 2 to about 10 and most preferably from about 3 to about 10 indentations.

The shape and dimensions of the protrusions 11 and the indentations 13 are variable and may be selected to meet one or more of the following: enhance the ability of the composition to fill the entire boundary 59 readily, or to provide sufficient volume through which water or moisture on the substrate may move. The terminal points 59 may be at the intersection of portions 54 (e.g., linear regions or segments of the boundary 49. While the portions 54 are shown in the Figure as linear, alternatively, some curvature may exist around the terminal points 52.

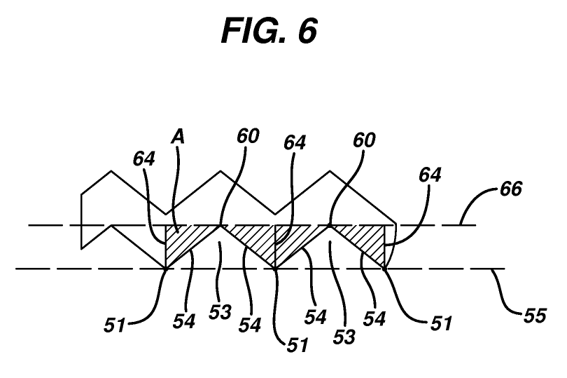

FIG. 6 again depicts the orifice 7 of FIG. 5, showing additional features thereof useful for determining a perimeter-to-area ratio. The perimeter-to-area ratio of orifice 47 is calculated by tracing the boundary 59 of the orifice 47, projected in the plane normal to the direction of flow of the composition therethrough (i.e., the plane of the paper). Lines 64 normal to imaginary line 55 are drawn through terminal points 51. A second imaginary line 66 is drawn parallel to imaginary line 55 such that it just above all of apices 60 of indentations 53. The area, A, bounded by portions 54 of the boundary 49, lines 66, and lines 64 (cross-hatched in FIG. 3) represent the area used to determine the perimeter-to-area ratio. The total length of that portion of the boundary 59 of the orifice 47 that both defines the indentations 13 and represents a border of area A (i.e., the segments 22 shown in FIG. 3) is the perimeter, P. Perimeter, P divided by area, A is the perimeter-to-area ratio.

The Applicants have found that, according to one embodiment, the boundary of the orifice has a perimeter-to-area ratio is large enough to facilitate the formation of channels in the composition urged therethrough. For example, the orifice may have a perimeter-to-area ratio that is from about 1 cm⁻¹ to about 10 cm⁻¹, preferably from about 0.4 cm⁻¹ to about 8 cm⁻¹, more preferably from about 1 cm⁻¹ to about 7 cm⁻¹, and most preferably from about 1.3 cm⁻¹ to about 6 cm⁻¹. The orifice of FIGS. 5 and 6, as calculated via the method described above has a perimeter to area ratio of about 1.8 cm⁻¹.

In order to provide sufficient volume through which water or moisture on the substrate may move, the indentations may have a height 50 (the maximum distance from a point on line 55 to a point just touching the boundary 59, shown in FIG. 5) of from about 0.5 mm to about 5 mm, preferably from about 0.5 mm to about 3 mm, more preferably from about 0.75 mm to about 2 mm.

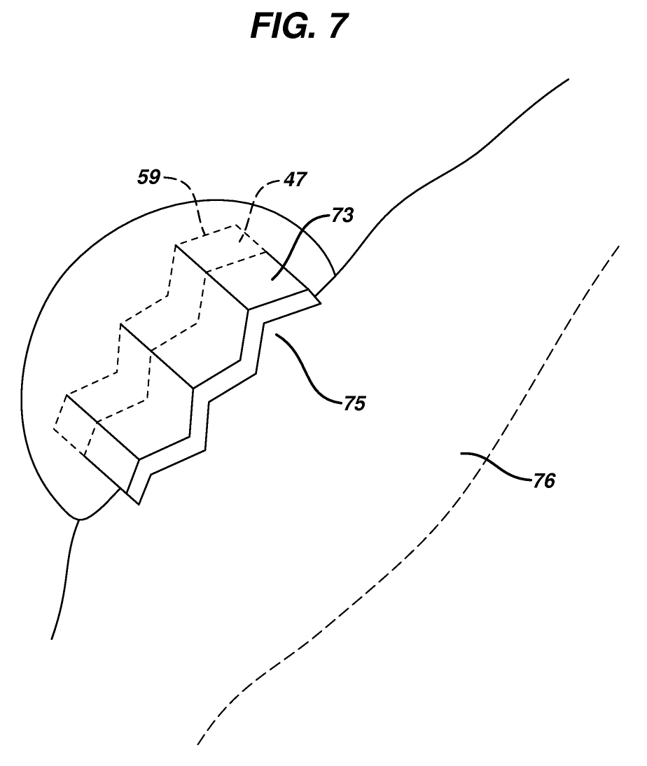

Referring to FIG. 7, composition exiting the orifice 47 forms an extruded portion 73 that sufficiently conforms to the boundary 59 (shown in phantom in FIG. 7) and thereby adopts, in its cross-section, the shape of the boundary 59 of the orifice 47. For at least some period of time, the extruded portion 73 maintains this shape. As such, expressing the composition from the orifice 47 effects the formation of plurality of channels 75 in the extruded portion 73. The plurality of channels 75 are substantially analagous to the indentations 53 in the boundary 59 of the orifice 47. The substrate 76, contacted with the extruded portion 73, is inclined to come into contact with it such that the plurality of channels 75 are disposed between the extruded portion 73 and the substrate 76. Furthermore, since the composition has the ability to remember the shape of the boundary 59, the extruded portion 73 is inclined, for a period of time, to remain in contact with the substrate 76 in the same orientation, i.e., such that the plurality of channels 75 are disposed between the extruded portion 73 and the substrate 76.

As described above, the composition extruded from the orifice 47 “remembers” the shape of the boundary 59 for a period of time. This period of time, i.e., “memory period” or “relaxation time” is generally at least about 2 seconds, preferably at least about 5 seconds. In certain embodiments, the relaxation time is at least about 1 minute, and, in other preferred embodiments, for at least about 3 minutes. As will be recognized by those of skill in the art, the relaxation time (time constant describing the rate of relaxation of the stress on the material that has been deformed to a certain strain) may be measured using standard procedures known in the art. Preferably, the relaxation time is measured by shear stressing the material to be measured to above the yield value so it flows as a liquid i.e. typically 20 to 100 pascals for structured fluids. The fluid will then flow and the relaxation time may be measured.

In order to effect this degree of memory into the composition, in one embodiment the composition is a “structured composition,” i.e., composition having a Yield Stress from about 1 Pascal (Pa) to about 1500 Pa as measured via the “Yield Stress Test” described in the Test Methods below. Examples of certain preferred structured compositions include those having a Yield Stress of from about 1 Pa to about 1500 Pa, preferably from about 10 Pa to about 1100 Pa, as measured by the Yield Stress Method described hereafter. In certain embodiments, the composition preferably includes one or more of lamellar, spherulitic, liquid-crystal and/or optically birefringent phases phase that is largely composed of one or more surfactants that is dispersed within an exterior (typically aqueous) phase. The viscosity of the personal care composition may be such that the composition is spreadable such as that of a cream or lotion or gel. For example, when measured using a LVT3 spindle at 30 rpm, the viscosity may be from about 500 cps to about 2000 cps.

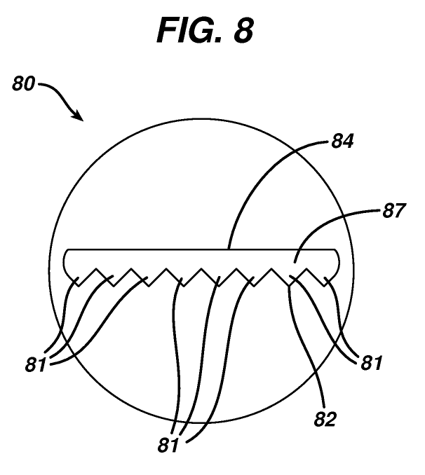

While various orifices shapes and designs are contemplated for use in the instant invention, e.g., various geometric or aesthetic patterns may provide the required protrusions and indentations. Another of such embodiments is shown in FIG. 8. Container 80 of FIG. 5 includes an orifice 87 that has eight protrusions 81 along a serrated edge 82. Orifice 87 also includes a flat edge 84. Flat edge 84 is shown for two reasons: one to illustrate that although flat edge has no protrusions and no indentations, orifice 87 is still consistent with embodiments of the invention, since serrated edge 82 does have these features. Secondly as described in the Examples section, composition extruded from an orifice similar to orifice 107 was tested according to the Wet Slip test in order to compare results when the composition therein was purposefully extruded with the serrated edge 82 against wets skin versus the situation in which the same composition was purposefully extruded with the flat edge 84 against wets skin. For clarity, flat edge 84 could be omitted and replaced with a serrated edge similar to serrated edge 82. This alternative embodiment would also be consistent with embodiments of the invention.

Products of the present invention are typically used for topical application to the body or another surface. Depending upon the particular function, the composition within the product of the present invention may be rinsed with water or rubbed onto the skin and allowed to remain without rinsing. Preferably, the compositions of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition. Particularly suitable uses for compositions of the present invention include body washes and conditioners as well as hair shampoos and conditioners, and facial cleansers. Compositions of the present invention may also be used for cleansers with acne-treatment benefit agents, stress-relief compositions (e.g., compositions with high concentrations, such as greater than about 1%, such as greater than about 3% of fragrant compounds), among other personal care applications.

In certain embodiments, the compositions produced via the present invention are preferably used as or in personal care products for cleansing, treating, or delivering active ingredients to least a portion of the human body. Examples of certain preferred personal care products include various products suitable for application to the skin, hair, and/or vaginal region of the body, such as shampoos, hand, face, and/or body washes, bath additives, gels, lotions, creams, and the like. As discussed above, applicants have discovered unexpectedly that the instant methods provide cost-effective personal care products having good aesthetics, and in certain embodiments one or more of desirable properties such as foaming characteristics, rheology, foam, and high surfactant loading.

The present invention provides methods of treating and/or cleansing the human body comprising contacting at least a portion of the body with a composition of the present invention. Certain preferred methods comprising contacting mammalian skin, hair, eyes, and/or vaginal region with a composition within a product of the present invention to cleanse such region and/or treat such region for any of a variety of conditions including, but not limited to, acne, wrinkles, pigmentation, eczema, dermatitis, diaper rash, sunburn prevention, dryness, pain, itch, and the like. In certain preferred embodiments, the contacting step comprises applying a composition of the present invention to human skin, hair or vaginal region.

The cleansing methods of the present invention may further comprise any of a variety of additional, optional steps associated conventionally with cleansing hair and skin including, for example, lathering, rinsing steps, and the like.

The methods of the present invention may further comprise any of a variety of steps for mixing or introducing one or more of the optional components described hereinabove with the structured composition of the present invention either before, after, or simultaneously with the combining step described above. While in certain embodiments, the order of mixing is not critical, it is preferable, in other embodiments, to pre-blend certain components, such as the fragrance and the nonionic surfactant before adding such components into the structured composition.

### EXAMPLES

The following Yield Stress Test is used in the instant methods. In particular, as described above, the Yield Stress test is used to determine whether a composition is structured, according to the present invention. Furthermore, the Degree of Heaping Test may be used to determine the ability of the composition to recover shape rapidly.

### Yield Stress Test:

The following Yield Stress Test is performed on various personal care compositions to determine the Yield Stress according to the present invention. Samples are placed in a water bath set at 25° C. for a period time sufficient to allow the sample to equilibrate (at least about an hour). The procedure is accomplished by gently placing about 1.0 grams of the composition to be tested was on the base plate of a properly calibrated rheometer (e.g., Advanced Rheometer AR 2000) having a 20 mm cone with a 1 degree angle, a 20 mm plate, a water bath, and a solvent trap. The sample size is just sufficient to allow some minor flow of the sample out of the gap once the final position of the cone and plate was reached (0.030 mm). To minimize shearing of the sample prior to testing, each sample is applied to the plate in a consistent manner, by gently scooping out the sample in one motion without significant shear or spreading, evenly layered on the plate, and without compressing and rotating the spatula away from the sample. The sample is centered on the base plate and laid relatively even across the plate. Once the measurement position is reached, a small bulge of the sample material protruded from the gap. This was removed quickly and gently so as not to disturb the top plate and pre-shear the sample. [If the top plate was moved then the run is aborted.] The sample preparation described thus far is less than 20 seconds to reduce undue drying of the sample. The instrument is set for a controlled shear rate run (log) with a shear rate spanning from 0.01⁻¹, to 300⁻¹; 300 data points collected; 300 seconds test duration; 25° C. water bath. The output device attached to the rheometer is set to plot stress (Pa) as a function of shear rate s⁻¹. Yield stress is determined from the plot of yield stress versus shear rate as the stress at which the curve departs from linearity. The average and standard deviation of the 3 runs is determined.

### Degree of Heaping Test:

The following Degree of Heaping Test is performed on various personal care compositions to determine the H-B Dimension according to the present invention.

Immediately after completing the Yield Stress Test above, the cone is then removed from the plate using the automated lift motor on the rheometer. The sample is left on the plate for 30 minutes and a digital picture is taken with a Canon S25, 5 megapixel camera. The picture is evaluated using a box counting technique method, starting with a box scale of one box covering the plate sample area and doubling the number of boxes with each iteration until the number of boxes equals one thousand and twenty four. The H-B dimension of the material is calculated by plotting log N(l) versus log l, where N(l) is a number of boxes containing any surface of the material and l is a resolution representing the reciprocal of the number of the boxes (i.e., box resolution) and wherein the H-B dimension is a straight line slope of the plot from eight boxes to one thousand and twenty four boxes. The test method repeated so that 10 replicates were performed for each sample. If the resulting H-B dimension has a relative standard deviation of less than 10% the value is reported for the sample. In certain embodiments of the invention, the structured composition has an H-B dimension of less than about 1.7, preferably less than about 1.6, more preferably less than about 1.5, and even more preferably less than about 1.4

### Wet Slip Test:

The following Wet Slip Test is used in the instant methods and in the following Examples. In particular, as described above, the Wet Slip Test is used to characterize the ability of the product to resist slipping along a wet or moist surface such as skin. The following Wet Slip test was performed on various personal care products. The palm of a standard subject's hand was held under water for two seconds and then placed with its base against the countertop and held perpendicular thereto. Within six seconds the product to be tested by urging the composition of the product through its nozzle and applying the composition by contact to the hand. For Comparative Examples C1 and C2 and Ex. 1, the length of the extruded composition was fixed at 1.5 inches (3.81 cm). For other examples, the length of the extruded body was varied. Two minutes were allowed to pass and the distance that the extruded composition fell downward was recorded.

### Comparative Examples C1-C2 and Inventive Example Ex. 1

A structured composition, S1 was prepared by blending the following ingredients according to the materials and amounts listed in Table 1:

TABLE 1

| INCI Name/Ingredient | Trade Name | % wt/wt |
|---|---|---|
| Water | Water | 26.55 |
| Glycerin | Emery 917 | 1.000 |
| Cyanopsis Tetragonologa Gum | Jaguar S | 0.250 |
| Sodium Coco-sulfate | Mackol CAS 100-N | 1.500 |
| Cocamidorpopyl betaine (32%) | Tegobetaine L-7V | 32.00 |
| Guar Hydroxytrimonium Chloride | Jaguar C17 | 0.250 |
| Disodium EDTA | Versene NA | 0.200 |
| Sodium Lauroamphoacetate (32%) | Miranol Ultra L-32 (32%) | 4.000 |
| C12-13 Alcohols | Isalchem 123A | 2.000 |
| Ammonium Lauryl Sulfate | Standapol A | 5.000 |
| Sodium Trideceth Sulfate (30%) | Cedapal TD-403 (30%) | 20.00 |
| Dimethicone | DC-200 (60,000) | 0.500 |
| Sunflower Oil | Florasun 90 | 3.000 |
| Petrolatum | White Petrolatum | 1.000 |
| Titanium Dioxide | Titanium Dioxide | 0.300 |
| Fragrance | Fragrance | 1.500 |
| DMDM Hydantoin & IBC | Glydant Plus | 0.450 |
| Citric Acid | Citric Acid (50% soln) | 0.500 |
| Total | | 100.00 |

The structured composition noted in Table 1 was prepared as follows: Ingredients were added in the order listed to a suitable size vessel equipped with an overhead propeller type mixer. Agitation was sufficient to maintain good batch movement without aeration. Components were added while maintaining constant agitation. pH was measured after the last component was added and adjusted to 5.5-6.5 Citric acid was then added to reduce to pH to between about 6.0 and 6.5. Examples Ex. 1 and C1 and C2 were evaluated for Wet Slip, using the Wet Slip Test described above, with values in inches (in) reported in Table 2.

Comparative Example C1 was prepared by placing the structured composition, S1 described above in a container having a star-shaped orifice having only one indentation, a perimeter-to-area ratio of about 1.2 cm⁻¹ and an area of about 0.23 cm².

Comparative Example C2 was prepared by placing the structured composition, S1 described above in a container having an orifice similar to FIG. 8, with the flat side purposefully positioned against the skin. Thus, the orifice of FIG. 8, while it actually had multiple indentations, and is consistent with embodiments of the invention described herein, the orifice as tested had no indentations and is meant to show comparative performance. The orifice had a perimeter-to-area ratio of about 2.2 cm⁻¹ and an area of about 0.1245 cm².

Inventive Example Ex. 1 was prepared by placing the structured composition, S1 described above in a container having an orifice similar to FIG. 8, with the serrated side purposefully positioned against the skin. Thus, the orifice, as tested had 7 indentations and eight protrusions and a perimeter to area ratio of about 5.2 cm⁻¹ and an area of about 0.1245 cm².

Three experiments were conducted to compare the Wet Slip of the inventive products to conventional products. Results for Wet Slip resistance are reported in Table 2, below.

TABLE 2

| | | Wet Slip |
|---|---|---|
| Example | Description | (in.) |
| Ex. 1 | Composition of Example S1 through | 0 |
| | orifice of FIG. 8 by positioning the serrated | |
| | side of orifice against the skin (seven | |
| | indentations P/A = 5.2; A = 0.1245) | |
| C2 | Composition of Example S1 through | 0.35 |
| | orifice of FIG. 8 by positioning the flat | |
| | side of orifice against the skin (no | |
| | indentations, P/A = 2.2; A = 0.1245) | |
| C1 | Composition S1 through star-shaped orifice | 0.54 |
| | (one indentation; P/A = 1.2; A = 0.23) | |

As shown in the Table, much less wet slip of the structured composition S1 occurs when the composition is extruded so that a plurality of channels are disposed between the composition and the wet skin, when compared to extruding the composition such that no channels are present therebetween.

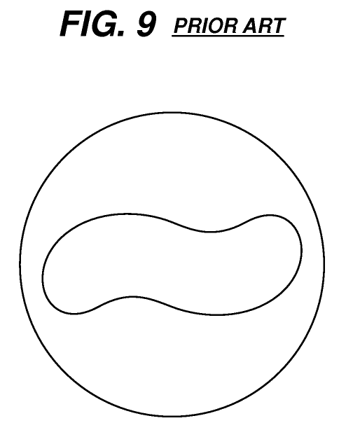

In another experiment, the composition S1 was separately extruded either through (a) the orifice of FIG. 8 by positioning the serrated side of orifice against the skin, as described for Inventive Example Ex. 1, or (b) the orifice of commercially available OLAY Body Wash Plus Radiance Ribbons, 10 fl. oz, (available from Procter and gamble of Cincinnati, Ohio). The OLAY product had a single-wave-shaped orifice having only one indentation, essentially as show in FIG. 9. The OLAY orifice had a cross-sectional area of about 0.55 cm². In this second experiment, the length (and therefore the mass of the extruded body) was allowed to vary. In this manner, one controls for variations in mass, and the influence mass may have on the extruded body's tendency to slip. The weight-normalized wet slip (WSₙ) is calculated by dividing wet slip by the mass of extruded body.

| | | Mass | Wet Slip | WSₙ |
|---|---|---|---|---|
| Example | Description | (g.) | (in.) | (in/g) |
| | OLAY Body Wash orifice | 3.2 | 63 | 20 |
| | OLAY Body Wash orifice | 2.2 | 46 | 21 |
| | OLAY Body Wash orifice | 1.1 | 16 | 15 |
| | OLAY Body Wash orifice | 2.6 | 24 | 9.2 |
| | OLAY Body Wash orifice | 3.6 | 18 | 5 |
| | OLAY Body Wash orifice | 3.5 | 30 | 8.6 |
| | OLAY Body Wash orifice | 1.2 | 31 | 26 |
| | Orifice of FIG. 8-serrated | 2.9 | 9 | 3.1 |
| | side | | | |
| | Orifice of FIG. 8-serrated | 2.8 | 15 | 5.4 |
| | side | | | |
| | Orifice of FIG. 8-serrated | 2.8 | 15 | 5.4 |
| | side | | | |
| | Orifice of FIG. 8-serrated | 0.9 | 0 | 0 |
| | side | | | |
| | Orifice of FIG. 8-serrated | 2.2 | 4 | 1.8 |
| | side | | | |
| | Orifice of FIG. 8-serrated | 2.2 | 3 | 1.4 |
| | side | | | |

Thus, for a given mass of the composition S1, much less wet slip occurs when the composition is extruded through the orifice if FIG. 3 when compared with the OLAY prior art orifice.

We claim:

1. A personal care product comprising:
a container suitable to be held in a user's hand, the container comprising an orifice, wherein the orifice defines a shape that includes a plurality of protrusions and a plurality of indentations;
a structured composition within said container, wherein said composition, when extruded through said orifice, forms an extruded portion of structured composition comprising a plurality of contact surfaces for contacting a body surface and a plurality of channels intermediate said contact surfaces.

2. The product of claim 1 wherein said orifice defines a boundary having an area of from about 0.005 cm² to about 20 cm².

3. The product of claim 1 wherein said orifice defines a boundary having an area of from about 0.04 cm² to about 0.5 cm².

4. The product of claim 1 wherein said orifice defines a boundary having an area of from about 0.005 cm² to about 0.04 cm².

5. The product of claim 1 wherein said orifice defines from 2 to about 20 indentations.

6. The product of claim 1 wherein said orifice defines from 2 to about 10 indentations.

7. The product of claim 1 wherein said orifice defines from 3 to about 10 indentations.

8. The product of claim 1 wherein said orifice defines a boundary having a perimeter-to-area ratio of from about 1 cm⁻¹ to about 10 cm⁻¹.

9. The product of claim 1 wherein said composition exhibits a WSₙ of less than about 5 inches per gram upon extrusion onto the body surface.

10. The product of claim 1 wherein each of the indentations is substantially the same size and shape as the other indentations defined by said orifice.

11. The product of claim 1 wherein said orifice defines at least two indentations that differ in size, shape, or both.

12. The product of claim 1 wherein said composition has a relaxation time of at least about 1 minute.

13. The product of claim 1 wherein said composition comprises one or more surfactants and at least one structuring agent.

14. The product of claim 1 wherein said product comprises a cleanser for skin.

15. The product of claim 1 wherein said product comprises a composition for application to the eye.
